(19) United States
(12) Patent Application Publication
(10) Pub. No.: US 2006/0165645 A1
(43) Pub. Date: Jul. 27, 2006

(54) Lipid preparation, particularly cosmetic preparation

(76) Inventors: Simona Lebok, Nurnberg (DE); Wolfgang Winkler, Lauf (DE)

Correspondence Address: BACHMAN & LAPOINTE, P.C., 900 CHAPEL STREET, SUITE 1201, NEW HAVEN, CT, 06510, US

(21) Appl. No.: 10/525,637

(22) PCT Filed: Aug. 7, 2003
(86) PCT No.: PCT/EP03/08793

§ 371 Date: Feb. 22, 2005

(30) Foreign Application Priority Data
Aug. 31, 2002 (DE) 102 40 322.8

(51) Int. Cl. A61K 36/899; A61K 8/97

(52) U.S. Cl. 424/74; 424/769; 424/750

(57) ABSTRACT

A lipid-bearing preparation and method for preparing same in particular in the form of a stick or a workable paste, which is suitable for cosmetic uses, in particular in the field of decorative cosmetics for coloring and improving the appearance of the skin, the lips and the eyelids. The lipid-bearing preparation is in water-free form and contains in the lipid phase exclusively vegetable-base raw materials.

### BACKGROUND OF THE INVENTION

The invention concerns a lipid-bearing preparation, in particular in the form of a stick or a workable paste, which is suitable for cosmetic uses, in particular in the field of decorative cosmetics, for coloring and improving the appearance of the skin, the lips and the eyelids. Mention may be made here by way of example of lip rouge and blusher, lipliner, eyeliner, eyebrow pencils, makeup, covering pencil, concealer or eyeshadow. It can also be used as lip care stick, lip balm, lip gloss, as a fixing base for the lips, as a foundation for skin care or as a sun-protection agent. That lipid-bearing preparation is in particular in water-free form.

Preparations of the indicated kind usually contain lipids, such as for example fats, oils, oil-soluble vegetable extracts and medium-chain to long-chain fatty acids and waxes which can be of vegetable or animal origin, which can be derived from mineral sources such as for example petroleum oil or which were obtained by synthesis or chemical modification of the specified substances. It is also known to use silicon-organic compounds such as for example dimethicone, phenyltrimethicone, diphenyldimethicone, volatile cyclomethicones, silicone waxes and the like.

In addition they may contain a solid phase consisting of finely divided fillers and coloring agents. In the case of sun-protection agents, it is possible to use particularly finely divided pigments, so-called nanopigments, involving an average particle size of between 5 and 25 nm, which act transparently on the skin and no longer color it. Mention may be made here by way of example of silicon dioxide, titanium dioxide and zinc dioxide.

Products derived from petroleum oil are not unlimitedly available as they originate from finite sources. In addition there are recommendations from a dermatological point of view, for example from the SCF, that as a precaution no petroleum oil-based raw materials should be used in the lip region in order to avoid accumulation thereof in the gastrointestinal tract. Therefore vegetable raw materials which re-grow are to be preferred thereto. Vegetable raw materials also do not involve the questions which arise out of intensive livestock farming with subsequent use of animals.

DE 199 40 221 discloses for example a body care agent which is in the form of a free-standing stick. So that the stick retains its shape it must be of a quite specific structure, in which respect it comprises an inner core with cosmetic ingredients and is encased by a layer which is made up of proteins, polyols, softening agents and optionally cosmetic additives. The predetermined structure means that a plurality of working steps are required for manufacture.

DE 107 07 309 discloses a solid cosmetic composition which is made up from various solidified cosmetic oils. In the examples mixtures of animal and synthetic oils are used for that purpose. The disadvantage of that composition is that only very small amounts of pigments can be used, which are in a range of between only 0.1 and 4% by weight.

German patent DE 304 76 49 describes a cosmetic composition in emulsion form comprising a mixture of oils and the non-saponifiable ingredient of an oil. The composition is neither intended for nor is it suitable for being formulated in the form of a stick or paste.

In European patent application EP 0 667 146 the inventors set themselves the object of providing lip makeup in the form of a soft paste which can be applied by means of an applicator and they attained that object by providing a composition containing a wax in a fat phase, the dynamic viscosity being adjusted in a predetermined range. Preferred fat ingredients in that case are components originating from petroleum oil such as paraffin oil, vaseline, mineral oil, the use of which is precisely to be avoided with the present invention.

A publication which is concerned with a water-bearing composition for a lipstick is EP 0 522 618. A disadvantage with water-bearing compositions is the higher level of susceptibility to bacterial contamination and thus the lower level of stability.

A composition comprising an oil phase and a pigment is known from U.S. Pat. No. 6,013,122. It will be noted however that this involves a liquid tattoo ink which does not permanently color but is removable. That composition is not intended to be processed to form sticks or pastes.

In order to make an oil accessible to processing in cosmetic materials, a refining process is required. A specific embodiment in that respect is described in U.S. Pat. No. 5,653,966.

A composition which has a high content of multiply unsaturated fatty acids is known from U.S. Pat. No. 5,445,822, which is to be processed to form a cosmetic preparation, in particular an emulsion. A disadvantage of such a composition is the low level of stability thereof.

The inventors of WO 02/38109 discovered that supercritical fluid extraction makes it possible to extract from Rubus chamaemorus ingredients which are advantageous as an addition to cosmetic compositions. The manufacture of such cosmetic products however is fairly complicated and expensive.

In regard to the manufacture and processing of lipid-bearing compositions of the specified kind it is to be borne in mind that they can be subjected over a prolonged period of time to relatively high temperatures in the region around between 70 and 110° C.—on the one hand if the raw materials are melted and homogenised together with coloring agents and other additives and on the other hand if they are put into their definitive form. In that case the raw materials used are not to experience chemical modification, or are to change chemically only to a very limited extent, due to the action of heat and oxygen in the air. Vegetable raw materials, particularly when they contain double bonds or conjugate double bonds in the carbon chain, have a tendency to experience rearrangements, additions, peroxide formation and the like, which can result in negative odorous changes. Such changes can be induced or also speeded up in the sense of a catalyst action due to the presence of certain pigments. Hitherto, only a few vegetable raw materials are known, which can be processed to afford lipid-bearing preparations of the specified kind with a satisfactory result and without adverse effects on quality.

Therefore the object of the invention was to provide a lipid-bearing preparation, in particular in the form of a stick or a workable paste, which is suitable for cosmetic uses, in particular in the field of decorative cosmetics, for coloring and improving the appearance of the skin, the lips and the eyelids, which can be easily applied, which has good adhesion and lasts for a long time and which does not migrate, or migrates only to a minimal extent, from the original location of application into the immediate surroundings. This preparation is to be based on purely vegetable and vegetable-based raw materials which do not suffer from the above-indicated disadvantages, but otherwise is to be as free as possible of animal raw materials, petroleum oil-based raw materials, in particular in order to avoid the known disadvantages thereof in the immediate area of the eyes. In addition the aim is to provide that it is possible to forego the use of silicone oils or silicone derivatives. If the preparation is in stick form the aim is that it should be stable in respect of storage at different storage temperatures which can arise on the varying transportation routes and at the user himself and—if it is in the form of a workable paste—it is not to exhibit any syneresis effects after prolonged storage.

In comparison with products in accordance with the state of the art therefore the aim is that this preparation can be applied softly and gently and in workable fashion, it does not become tight on the skin, the lids and the lips and dry them out, it has good durable adhesion, as far as possible it does not transfer on to articles and textiles or other regions of the skin and in the immediate proximity of the eyes it also does not lead to irritations or other adverse sensations.

### SUMMARY OF THE INVENTION

The object is attained in that there is provided a lipid-bearing preparation which is made up exclusively from vegetable or vegetable-based raw materials which are at least in part to involve hydrogenated vegetable or vegetable-based raw materials.

It was surprisingly found that, by virtue of its composition, the preparation according to the invention is suitable for being cast to form pencil or stick leads, even with pigment contents of between 40 and 50% by weight, so that sticks can be easily made from the material according to the invention. That was hitherto not possible with the known materials, using vegetable ingredients. In addition the invention provides particularly stable preparations.

### DETAILED DESCRIPTION

In particular in accordance with the invention there is provided a lipid-bearing preparation which has at least one oil phase and a solid phase. In that respect the oil phase is made up exclusively from vegetable or vegetable-based raw materials.

The raw materials are designated using the designations known to the persons skilled in the relevant art, in accordance with the ‘International Nomenclature of Cosmetic Ingredients’ (referred to as ‘INCI names’). The following may be mentioned here by way of example: hydrogenated vegetable oils such as for example hydrogenated jojoba oil hydrogenated cottonseed oil, hydrogenated vegetable oil, hydrogenated rapeseed oil, hydrogenated castor oil, hydrogenated coco-glycerides and similar, as well as Magnifera indica (mango seed oil), Limnanthes alba (meadowfoam seed oil), Butyrosperum parkii (shea butter), Macadamia ternifolia nut oil (macadamia nut oil), Buxus chinensis (jojoba oil); waxes such as carnauba wax, candelilla wax, japan wax (Rhus succedanea), rice wax, sugar cane wax and similar.

In that respect the content of hydrogenated vegetable oils in the lipid-bearing preparation is between 5 and 80% by weight, preferably between 15 and 50% by weight. The content of vegetable waxes is between 0.1 and 30% by weight, preferably between 1 and 20% by weight. The content of vegetable oils including jojoba oil (Buxus chinensis) which involves in the chemical sense a liquid wax is in a range of between 1 and 65% by weight, preferably between 5 and 45% by weight. It will be appreciated that the specifications in respect of the amounts which are used and which are preferably used are with the proviso that the total thereof is finally made up to 100% by weight.

A lipid-bearing preparation of the specified kind has proven to be particularly advantageous, which contains a combination of hydrogenated jojoba oil and Limnanthes alba (meadowfoam seed oil). Hydrogenated jojoba oil is commercially available and can be obtained in various levels of hydration and thus with varying levels of hardness—measured for example by needle penetration—and with varying melting points. By suitably mixing various commercially available types of product the man skilled in the relevant art can find out the mixture which is most suited to his purposes and can thus possibly adjust the end product in the optimum fashion, for example in respect of consistency and viscosity.

The amounts used of hydrogenated jojoba oil and Limnanthes alba (meadowfoam seed oil) should be in that respect in each case between 2 and 35% by weight and preferably between 5 and 25% by weight and should be used relative to each other in a ratio of between 1:2 and 2:1. Limnanthes alba (meadowfoam seed oil) is considered to be a particularly oxidation-stable and rancidity-stable vegetable oil—it was possible for that product property to be still further increased by the combination with hydrogenated jojoba oil so that the preparations according to the invention showed themselves to be surprisingly stable in respect of storage, even at elevated storage temperatures. However they are not only highly oxidation-stable but also the olfactory properties do not change over a prolonged storage time. The lipid-bearing preparations according to the invention can be used unchanged in temperature ranges of about 42° C. and are storage-stable at up to about 52° C. They are also light, workable and can be uniformly applied, they have good adhesion and last for a long time without spreading on the skin to a substantial extent. Thus it is for example also possible to produce from a lipid-bearing preparation of the specified kind, cosmetic pencils with cast leads which can involve a lead diameter in the range of between 2 and 6 mm, with a pigment and solid content in a range of between 1 and about 50% by weight, preferably between 5 and 40% by weight, quite particularly preferably between 10 and 30% by weight, with respect to the total amount of the preparation. Such cosmetic pencils which, with pigment contents around between 40 and 50% by weight were hitherto preferably produced by an extrusion method can be used in particular as lipliner, eyeliner or eyebrow pencils, because of their good coverage capability. The use of the above-mentioned combination of hydrogenated jojoba oil and Limnanthes alba (meadowfoam seed oil) means that lipid-bearing preparations of the specified kind with high solid contents of up to 50% by weight are accessible to the modern casting methods—in principle however it is possible for such lipid-bearing preparations also to be extruded in accordance with the known methods and then subjected to further processing with the methods which are also known to afford wood-encased cosmetic pencils.

The second essential ingredient of the preparation according to the invention is a solid phase which can comprise fillers and/or pigments. The proportion of the solid phase can be adjusted depending on the respectively desired consistency and desired effect of the material. Thus the proportion of the pigment depends inter alia on the desired color and the proportion of fillers depends on the desired consistency of the material. It is also possible to incorporate effect agents such as glittery or luminescent ingredients.

Examples of substances which can form the solid phase are for example fillers such as for example talcum, kaolin, starch and modified starch, polytetrafluoroethylene powder (Teflon), nylon powder, boronitride, insoluble metal soaps such as Mg stearate, Ca stearate, Sr stearate, Zn stearate and inorganic or organic pigments. The latter may be mentioned by way of example: titanium dioxide, zinc oxide, iron oxides, chromium oxide, chromium hydroxide, ultramarine, Berlin blue (ferric blue), mica, pearl gloss agents such as for example mica coated with titanium dioxide, colored mica coated with titanium dioxide and metal oxides, bismuth oxide chloride, coated bismuth oxide chloride, metal powder in flake form of aluminum, brass, bronze, copper, silver, gold and laking means of organic coloring agents with aluminum, barium, calcium or strontium. That list is only given by way of example and is not definitive. Those additives are implemented with the proviso that they are also approved by the respective national or regional cosmetic legislation.

The amount of the solid phase preferably depends on the maximum amount which is regulated in the individual countries by cosmetic legislation.

In that respect the quantitative proportions of pigments are in a range of between 1 and 50% by weight, preferably in a range of between 5 and 40% by weight and quite particularly preferably in a range of between 10 and 30% by weight. If the lipid-bearing preparation according to the invention is used as a light-protection agent it is possible to add thereto titanium dioxide and/or zinc oxide in the form of so-called ‘nanopigments’ with particle sizes in the range of between 5 and 25 nm in an amount of between 1 and 20% by weight, preferably between 5 and 15% by weight, optionally also in combination with conventional oil-soluble UV-A and/or UV-B light filter substances which are approved by the respective national or regional legislation. All those quantities in respect of the solid phase are also specified with the proviso that the total of all ingredients of the above-mentioned lipid-bearing preparation is made up to 100% by weight.

It is also possible to add to the preparation according to the invention the additives which are usual for cosmetics such as perfumes, anti-oxidants, preserving agents and the like in the amounts which are necessary for effectiveness and usual for cosmetic materials. It is preferred in that respect to have recourse to such substances which are of vegetable origin.

In that respect both inorganic and also organic pigments and organic dyes are to be considered as the coloring agents.

A further subject of the invention is also a method of producing the lipid-bearing preparation according to the invention.

As stated above by virtue of its particular properties the lipid-bearing preparation can be processed to form stick or pencil leads not only by extrusion but also by casting. The subject of the invention is therefore a method of producing a cosmetic pencil in which the lipid-bearing preparation according to the invention is formed into a lead by casting and then glued into wood in per se known manner and subjected to further processing to form pencils.

In a preferred embodiment in that respect the lipid-bearing preparation is cast at elevated temperature into a plastic casing sleeve of material which can be sharpened to a point, with a point being formed thereon. Methods for that purpose are known to the man skilled in the art.

By virtue of the particularly advantageous properties of the preparation according to the invention it is possible for the leads cast therefrom to be used in a rotary spindle mechanism. A further subject of the invention is therefore a method of producing a cosmetic pencil in which the lipid-bearing preparation according to the invention is cast at elevated temperature into a rotary spindle mechanism.

The preparation according to the invention can also be introduced in the form of a paste into tubes, pots and also cups.

The invention concerns lipid-bearing preparations, in particular in the form of a stick or pencil or a workable paste, which are applied to the skin, the semi-mucous membranes or in the proximity of mucous membranes, for example in the proximity of the eyes. In particular the lips are to be interpreted here as semi-mucous membranes. By way of example mention may be made of preparations for coloring of or for care of the lips, preparations for coloring of or for care of the skin such as for example makeup, rouge, camouflage for hiding age spots or rosacea, concealer, pencils for outlining lip contours, the contours of the eyes and the eyebrows and also sun-protection products with different sun protection factors (SPF), as far as so-called sun blocks, using very finely divided nanopigments or brightly colored, more highly pigmented sun blocks, which are popular for example with surfers and windsurfers as body paint.

The preparation can also be produced without the addition of coloring agents and may optionally contain so-called cosmetic active substances. It is then used as lip gloss or as a fixing agent which are applied over a lipstick. If that uncolored preparation contains light protection filter, it can be used as lip protection and lip care. As is known in contrast to the skin of the body the skin of the lips does not include any pigmentation. Suitable oil-soluble light filter substances which afford good protection in the UV-A and UV-B range are known in adequate numbers to the man skilled in the relevant art and are regulated by the respective national or regional legislation for example in the EU, in Japan and in the USA—in Germany for example by Appendix 7 to Regulation 3b of the Cosmetics Regulations and they are therefore not to be comprehensively listed here. Therefore mention will only be made by way of example of isoamyl p-methoxycinnamate as a UV-B filter, and 4-methylbenzylidene camphor as a UV-A filter.

The preparation according to the invention will now be described in detail by means of the Examples hereinafter, which however do not definitively describe the invention. In this respect all amounts are stated in percent by weight (% by weight) with respect to the total weight of the preparation:

Example 1 - Pasty lip rouge

| | |
|---|---|
| Hydrogenated jojoba oil | 11.000 |
| Limnanthes alba | 19.500 |
| Carnauba | 3.500 |
| Buxus chinensis | 31.550 |
| Butyrosperum parkii | 3.000 |
| Hydrogenated cottonseed oil | 9.500 |
| Pigments | 18.000 |
| Silica | 3.500 |
| Tocopherol | 0.350 |
| Ascorbyl palmitate | 0.100 |

Production is effected by a procedure whereby hydrogenated jojoba oil, Limnanthes alba (meadowfoam seed oil), carnauba, Buxus chinensis and hydrogenated cottonseed oil are put in a suitable homogenising machine with an anchor-type agitator and gear ring homogeniser and heated to about 90° C. The silica is then sprinkled in and dispersed by means of the homogeniser. The pigments are then added and the mixture is then homogenised under a high input of shearing force in order to destroy all pigment agglomerates. The mass is then deaerated by the application of vacuum. Then the anti-oxidants (tocopherol and ascorbyl palmitate) are added to the mixture while still hot and it is then subjected to brief post-homogenisation. The mixture is then cooled down further to about 35° C. with agitation with the anchor-type agitator. The mixture which is now pasty is transferred into the filling vessel and allowed to cool down to ambient temperature, without further measures. Then on a filling or assembly machine it is introduced into the appropriate vessels such as for example pots of glass or plastic material or bowls of metal after the preparation has been checked by quality assurance and passed. The result obtained is a workable soft paste of a viscosity of 5000 mPas.

Example 2 - Cream eyeshadow

| | |
|---|---|
| Candelilla cera | 2.500 |
| Carnauba | 1.800 |
| Hydrogenated castor oil | 3.500 |
| Hydrogenated jojoba oil | 9.500 |
| Limnanthes alba | 18.000 |
| Hyrogenated cottonseed oil | 4.000 |
| Buxus chinensis | 32.250 |
| Pigments | 9.000 |
| Mica (and) titanium dioxide | 16.000 |
| Silica | 3.000 |
| Tocopherol | 0.350 |
| Ascorbyl palmitate | 0.100 |

Production is effected in a similar manner to Example 1 but in this case firstly the pigments are added to the fat phase at about 90° C. and homogeneously worked thereinto, thereafter the pearl gloss agents are added and it is then briefly homogenised once again. The result obtained is a soft workable paste with a nice pearl gloss and a viscosity of 4200 mPas.

Example 3 - Lipgloss

| | |
|---|---|
| Carnauba | 1.650 |
| Candelilla cera | 2.000 |
| Limnanthes alba | 16.000 |
| Hydrogenated jojoba oil | 8.750 |
| Magnifera indica | 6.000 |
| Macadamia ternifolia nut oil | 7.500 |
| Butyrosperum parkii | 2.000 |
| Buxus chinensis | 44.550 |
| Rhus succedanea | 6.000 |
| Silica | 2.500 |
| Isoamyl p-methoxycinnamate | 1.500 |
| 4-Methylbenzylidene camphor | 1.000 |
| Tocopherol | 0.450 |
| Ascorbyl palmitate | 0.100 |

Production is effected substantially similarly to the above-described methods, wherein the light filter substances are added together with the tocopherol at about 45-50° C. The result obtained is an uncolored, transparent, very soft paste, with a viscosity of 2800 mPas, which can be introduced into tubes or pots.

Example 4 - Sun block for surfers

| | |
|---|---|
| Carnauba | 3.500 |
| Candelilla cera | 2.850 |
| Hydrogenated castor oil | 4.800 |
| Hydrogenated vegetable oil | 7.500 |
| Hydrogenated jojoba oil | 9.500 |
| Limnanthes alba | 16.000 |
| Buxus chinensis | 23.550 |
| Magnifera indica | 5.000 |
| Titanium dioxide (nanopigment) | 10.000 |
| Iron oxides (red and yellow) | 6.000 |
| Silica | 3.000 |
| Isoamyl p-methoxycinnamate | 4.500 |
| 4-Methylbenzylidene camphor | 3.000 |
| Tocopherol | 1.000 |
| Ascorbyl palmitate | 0.100 |

Production is effected in accordance with the method specified in Example 1. The result obtained is a red-orange solid preparation which is preferably suitable for being introduced into rotary spindle mechanisms. The sun protection factor (SPF) of that preparation is above 25.

Example 5 - Lipliner

| | |
|---|---|
| Hydrogenated jojoba oil | 34.000 |
| Limnanthes alba | 20.000 |
| Carnauba | 0.800 |
| Candelilla cera | 2.500 |
| Magnifera indica | 4.000 |
| Butyrosperum parkii | 1.000 |
| Silica | 8.300 |
| Chamomilla recuita | 1.000 |
| Tocopherol | 0.300 |
| Pigments | 28.000 |
| Ascorbyl palmitate | 0.100 |

Production of the lipliner is effected similarly to Example 1. The material is transferred into a casting machine and at about 85-90° C. either cast into plastic casing sleeves comprising a material which can be sharpened to a point, with a point being formed thereon, or is cast into metal casting molds and, after becoming cold, is removed from the mold and subjected to further processing again in known manner to form wood-encased cosmetic pencils.

Example 6 - Eyeliner

| | |
|---|---|
| Hydrogenated jojoba oil | 11.000 |
| Limnanthes alba | 19.500 |
| Hydrogenated vegetable oil | 4.500 |
| Buxus chinensis | 8.400 |
| Carnauba | 1.700 |
| Candelilla cera | 2.650 |
| Rhus succedanea | 4.500 |
| Magnifera indica | 4.000 |
| Macadamia ternifolia nut oil | 1.900 |
| Pigments | 30.000 |
| Talcum | 6.700 |
| Silica | 3.000 |
| Glyceryl caprylate | 1.000 |
| Chamomilla recutita extract | 0.800 |
| Tocopherol | 0.250 |
| Ascorbyl palmitate | 0.100 |

Production of the eyeliner is effected in a similar manner to the procedure set out in Example 5. The pigments which are particularly preferably used are inorganic pigments such as black, red and yellow iron oxides, titanium dioxide, ferric blue, ultramarine, chromium hydroxide green, chromium oxide green, manganese violet and mixtures thereof. It is optionally also possible to use pearl gloss pigments such as for example mica, mica coated with metal oxides, bismuth oxychloride, bismuth oxychloride coated with metal oxides, metals in flake form such as powder-form aluminum, bronze, brass, titanium, silver, gold or mixtures thereof, also in combination with inorganic pigments.

Example 7 - Eyebrow pencil

| | |
|---|---|
| Hydrogenated jojoba oil | 12.500 |
| Limnanthes alba | 10.500 |
| Rhus succedanea | 6.000 |
| Hydrogenated cottonseed oil | 6.000 |
| Carnauba | 2.200 |
| Candelilla cera | 2.600 |
| Butyrosperum parkii | 1.250 |
| Macadamia ternifolia nut oil | 2.200 |
| Buxus chinensis | 6.300 |
| Pigments | 35.000 |
| Talcum | 12.000 |
| Silica | 3.000 |
| Tocopherol | 0.350 |
| Ascorbyl palmitate | 0.100 |

Production is effected similarly to Example 1. After the material is completely cold the material is also passed twice by way of a three-roll mill, put into cartridge form in known manner and extruded in a conventional procedure to afford leads which are then glued into wood stick portions and subjected to processing to provide finished wood-encased pencils.

Lipid-bearing preparations which do not contain any water phase very often remain unpreserved in order as far as possible to exclude incompatibility reactions—which are comparatively very rare—on the part of lady consumers to preserving agents. In order however to exclude colonisation of the pencil surfaces with micro-organisms with subsequent re-contamination of the surface of the skin, it is possible to add to the lipid-bearing preparations according to the invention, vegetable-based preserving agents such as for example oil-soluble dry extract of rosemary or glyceryl caprate or mixtures of natural perfumes or perfumes which are identical to nature, with anti-microbial properties, such as for example geraniol, linalool, neroli, vanillin, eugenol, methyleugenol, palmarosa oil and the like in the desired and conventional amounts of between 0.05 and 1.0% by weight. Preferably the necessary amount of preserving agent is determined in a preservation loading test which is sufficiently known to the persons skilled in the art who are relevantly concerned with this aspect.

1-35. (canceled)

36. A lipid-bearing preparation for cosmetic uses, comprising an oil phase and a solid phase, wherein the oil phase comprises a mixture of vegetable-base raw materials.

37. A lipid-bearing preparation as set forth in claim 36, wherein the vegetable-base raw materials are selected from the group consisting of vegetable oils, hydrated vegetable oils, vegetable waxes and mixtures thereof.

38. A lipid-bearing preparation as set forth in claim 37, wherein the vegetable oils are selected from the group consisting of mango seed oil, meadowfoam seed oil, macadamia nut oil, shea butter, jojoba oil and mixtures thereof.

39. A lipid-bearing preparation as set forth in claim 37, wherein the hydrogenated vegetable oils are selected from the group consisting of hydrogenated cottonseed oil, hydrogenated vegetable oil, hydrogenated castor oil, hydrogenated coco-glycerides, hydrogenated jojoba oil and mixtures thereof.

40. A lipid-bearing preparation as set forth in claim 37, wherein the vegetable waxes are selected from the group consisting of canauba, candelilla cera, Rhus succedanea, rice wax, sugar cane wax and mixtures thereof.

41. A lipid-bearing preparation as set forth in claim 37, wherein the content of vegetable oils is in a range of between 1 and 65% by weight with respect to the total weight of the preparation.

42. A lipid-bearing preparation as set forth in claim 37, wherein the content of vegetable waxes is in a range of between 0.1 and 30% by weight with respect to the total weight of the preparation.

43. A lipid-bearing preparation as set forth in claim 42, wherein the content of hydrogenated vegetable oils is in a range of between 5 and 80% by weight with respect to the total weight of the preparation.

44. A lipid-bearing preparation as set forth in claim 37, wherein the content of vegetable oils is in a range of between 5 and 45% by weight with respect to the total weight of the preparation.

45. A lipid-bearing preparation as set forth in claim 37, wherein the content of vegetable waxes is in a range of between 1 and 20% by weight with respect to the total weight of the preparation.

46. A lipid-bearing preparation as set forth in claim 42, wherein the content of hydrogenated vegetable oils is in a range of between 15 and 50% by weight with respect to the total weight of the preparation.

47. A lipid-bearing preparation as set forth in claim 36, wherein the vegetable-base raw material comprises a mixture of hydrogenated jojoba oil and meadowfoam seed oil.

48. A lipid-bearing preparation as set forth in claim 47, wherein the amounts of hydrogenated jojoba oil and meadowfoam seed oil are each in a range of between 2 and 35% by weight with respect to the total weight of the preparation.

49. A lipid-bearing preparation as set forth in claim 47, wherein the amounts of hydrogenated jojoba oil and meadowfoam seed oil are each in a range of between 5 and 25% by weight with respect to the total weight of the preparation.

50. A lipid-bearing preparation as set forth in claim 47, wherein the ratio of the amounts used of hydrogenated jojoba oil to meadowfoam seed oil is between 1:2 and 2:1.

51. A lipid-bearing preparation as set forth in claim 36, wherein the solid phase is selected from the group consisting of fillers, inorganic pigments, organic pigments and mixtures thereof.

52. A lipid-bearing preparation as set forth in claim 51, wherein the fillers are selected from the group consisting of talcum, kaolin, starch, modified starch, polytetrafluoroethylene powder, nylon powder, boronitride, Mg stearate, Ca stearate, Sr stearate, Zn stearate and mixtures thereof.

53. A lipid-bearing preparation as set forth in claim 51, wherein the inorganic pigments are selected from the group consisting of titanium dioxide, zinc oxide, iron oxides, chromium oxide, chromium hydroxide, ultramarine, ferric blue, mica, mica coated with titanium dioxide, mica coated with titanium dioxide and with metal oxides, bismuth oxide chloride, coated bismuth oxide chloride, metal powder in flake form of aluminum, brass, bronze, copper, silver, gold and mixtures thereof.

54. A lipid-bearing preparation as set forth in claim 51, wherein the organic pigments are selected from the group consisting of Takings of organic coloring agents with aluminum, barium, calcium, strontium, zirconium and mixtures thereof.

55. A lipid-bearing preparation as set forth in claim 51, wherein the amount of organic and inorganic pigments are in a range of between 1 and 50% by weight with respect to the total weight of the preparation.

56. A lipid-bearing preparation as set forth in claim 51, wherein the amount of organic and inorganic pigments are in a range of between 5 and 40% by weight with respect to the total weight of the preparation.

57. A lipid-bearing preparation as set forth in claim 51, wherein the amount of organic and inorganic pigments are in a range of between 10 and 30% by weight with respect to the total weight of the preparation.

58. A lipid-bearing preparation as set forth in claim 55, wherein the pigments have particle sizes in the range of between 5 and 25 nm.

59. A lipid-bearing preparation as set forth in claim 53, wherein titanium oxide and/or zinc oxide used as light protection agent is present as nanopigments in an amount of between 5 and 25% by weight with respect to the total weight of the preparation.

60. A lipid-bearing preparation as set forth in claim 53, wherein titanium oxide and/or zinc oxide used as light protection agent is present as nanopigments in an amount of between 5 and 15% by weight with respect to the total weight of the preparation.

61. A lipid-bearing preparation as set forth in claim 59, wherein the nanopigments are used in combination with oil-soluble UV-A- and UV-B-light filter substances.

62. A lipid-bearing preparation as set forth in claim 61, wherein 4-methylbenzylidene camphor and isoamyl p-methoxycinnamate are used in combination with the nanopigments as UV-A- and UV-B-light filter substances.

63. A lipid-bearing preparation as set forth in claim 36, wherein the preparation is provided as at least one of lip rouge, blusher, lipliner, eyeliner, eyebrow pencil, eyeshadow, makeup, covering pencil, concealer, lip care pencil, lip balm, lip gloss, fixing foundation for the lips, care foundation for the skin and as sun protection agent.

64. A lipid-bearing preparation as set forth in claim 36, further comprising, as preserving agent, at least one of vegetable-based preserving agents, natural perfumes, perfumes which are identical to nature and mixtures thereof.

65. A lipid-bearing preparation as set forth in claim 64, wherein the preserving agent is present in an amount of between 0.05 and 1.0% by weight with respect to the total weight of the preparation.

66. A lipid-bearing preparation as set forth in claim 65, wherein the preserving agent comprises at least one of an oil-soluble dry extract of rosemary and glyceryl caprate.

67. A lipid-bearing preparation as set forth in claim 65, wherein the preserving agent contains perfumes which were selected from the group consisting of geraniol, linalool, neroli, vanillin, eugenol, methyleugenol, palmarosa oil and mixtures thereof.

68. A lipid-bearing preparation as set forth in claim 36, wherein the preparation is in the form of a stick glued into wood.

69. A lipid-bearing preparation as set forth in claim 36, wherein the preparation is in the form of a cast into a plastic casing sleeve.

70. A lipid-bearing preparation as set forth in claim 36, wherein the preparation is in the form of a stick, cast into a rotary spindle mechanism.

71. A lipid-bearing preparation as set forth in claim 36, wherein the preparation is in the form of a paste introduced into a tube or a pot.

72. A lipid-bearing preparation as set forth in claim 36, wherein the preparation is in the form of a paste introduced into a bowl of metal.

73. A method of producing a cosmetic pencil according to claim 36, wherein a preparation including shaping the preparation by casting to form a lead and further processed to form a pencil.

74. A method as set forth in claim 73, including casting into a rotary mechanism.

75. A method as set forth in claim 73, including casting into wood, glued and processed to form a pencil.

76. A storage stable cosmetic mass comprising a combination of hydrogenated jojoba oil and meadowfoam seed oil.
